(19) United States
(12) Patent Application Publication
(10) Pub. No.: US 2026/0126361 A1
(43) Pub. Date: May 7, 2026

(54) REMOTELY OPERATED GROUND TESTING APPARATUS AND METHOD

(71) Applicant: FNV IP B.V., Leidschendam (NL)

(72) Inventors: Peter LOOIJEN, Leidschendam (NL); Peter SALOME, Leidschendam (NL)

(73) Assignee: FNV IP B.V., Leidschendam (NL)

(21) Appl. No.: 19/117,601

(22) PCT Filed: Aug. 18, 2023
(86) PCT No.: PCT/EP2023/072787

§ 371 Date: Apr. 1, 2025

(30) Foreign Application Priority Data
Oct. 7, 2022 (NL) 2033255

(51) Int. Cl. G01N 3/303; G01N 3/02; G01N 33/24

(52) U.S. Cl. CPC G01N 3/303; G01N 3/02; G01N 33/24

(57) ABSTRACT

Increasingly high concentration of pollutants on the surface and in the ground is a continuous concern in today's world. Harmful substances endanger both land and water biodiversity and as well as endangers human health, particularly through food. Activities such as stock breeding and intensive farming use chemicals, pesticides and fertilizers pollute the land, e.g., heavy metals and other natural and man-made chemical substances. The present invention provides a remotely operated ground testing apparatus, a method for testing the ground, an automated launch and recovery system for use with the remotely operated ground testing apparatus, a container for containing and immobilizing the remotely operated ground testing apparatus, and a vessel comprising the automated launch and recovery system. Unlocking insights from Geo-Data, the present invention further relates to improvements in sustainability and environmental developments: together we create a safe and liveable world.

### FIELD OF THE INVENTION

The present invention relates generally to a remotely operated ground testing apparatus, and more particularly to a method for testing the ground, to an automated launch and recovery system for use with the remotely operated ground testing apparatus, to a container for containing and immobilizing the remotely operated ground testing apparatus, and to a vessel comprising the automated launch and recovery system. Unlocking insights from Geo-Data, the present invention further relates to improvements in sustainability and environmental developments: together we create a safe and liveable world.

### BACKGROUND OF THE INVENTION

Geotechnical surveys are commonly performed to obtain information on soil properties. This information is used in many applications ranging from site investigation prior to building onshore or offshore structures. It is often required to obtain soil samples or data on soil properties from different depths and in areas with limited accessibility. Obtaining soil data from all sorts of grounds, often with poor accessibility and/or where safety is at risk, often requires remotely operating systems.

Obtaining soil data from the seabed (i.e., the seabed) presents further challenges. At great water depths, it becomes necessary to build up a larger string to reach and penetrate the seabed. A string is the cable extending between a vessel and subsea equipment, which may aid in positioning, and data transmission.

Increasingly high concentration of pollutants on the surface and in the ground is a continuous concern in today's world. Harmful substances endanger both land and water biodiversity and endangers human health, particularly through food. Activities such as stock breeding and intensive farming use chemicals, pesticides and fertilizers pollute the land, e.g., heavy metals and other natural and man-made chemical substances.

Soil pollution is a well-known global threat that is particularly serious in regions like Europe, Eurasia, Asia, and North Africa, as indicated by the Food and Agricultural Organization of the United Nations (FAO). The FAO also affirms that both intense and even moderate degradation is already affecting one third of the world's soil. Moreover, recovery from such pollution is known to be very slow.

Phenomena such as erosion, loss of organic carbon, increased salt content, compacting, acidification and chemical pollution are the major causes of current soil degradation. The FAO distinguishes between two types of soil pollution:
Chemical pollution: mostly accounted for by specific causes, occurring in small areas. Land pollution such as this is normally found in cities, old factory sites, around roadways, illegal dumping grounds, and sewage treatment stations.
Widespread pollution: covers extensive areas and has several causes the reasons for which are difficult to identify. Cases such as these involve the spreading of pollutants by air-ground-water systems and seriously affect human health and the environment.

Among the most common causes of soil contamination caused by human activity, the FAO highlights industry, mining, military activities, waste—which includes technological waste—and wastewater management, farming, stock breeding the building of urban and transport infrastructures.

Accordingly, the need for providing sustainable techniques and methods to sample and test the soil has been increasing rapidly. Soil or ground investigations for detection of contamination is important in risk management analysis in infrastructure installation. This applies both on land and underwater. Boreholes may be drilled into the ground for many different purposes, such as obtaining core samples of the ground for risk assessment analysis in soil contamination, mineral exploration, scientific research, or geotechnical site investigations.

Ground investigations may however be particularly difficult to perform, for example in mountainous areas or in underwater landscapes where the ground profile is rather irregular, rough and/or unstable. When operating in regions where safety for operating crews is a concern, having efficient and reliable ground and seabed analysis techniques is a priority for the industry, specifically for companies and governments.

### BRIEF SUMMARY OF THE INVENTION

The present invention offers a reliable ground testing apparatus and method for testing all types of grounds and/or regions of the seabed, which can be easily, efficiently, and safely remotely operated. According to an aspect of the present invention, there is provided a remotely operated ground testing apparatus comprising:
a) a frame;
b) at least one ground penetrating testing tool;
c) at least one gripper, connected to the frame, to handle the at least one ground penetrating testing tool;
d) a storage unit, arranged to store the at least one ground penetrating testing tool and/or testing samples;
e) a connector for connecting the at least one ground penetrating testing tool to the ground testing apparatus;
f) at least three height-adaptable legs;
g) at least one environment detection sensor, arranged to provide environment information.

In the context of the present invention, soil can originate from any type of onshore and offshore grounds. In the context of the present invention, the term ground is to be understood as terrain on land and seabed or sea bottom underwater. The apparatus according to the present invention can be used to test and/or sample soil from onshore and offshore grounds, i.e., soils originating from land or a seabed. In the context of the present invention, the ground penetrating testing tools can be pipes, rods or other devices allowing ground penetration as well as testing of the ground and/or sampling of the ground/soil. For the avoidance of doubt, the apparatus according to the present invention penetrates the ground and tests and/or samples the composition of the ground, i.e., the soil. The terms sea or seabed can be understood in a broad sense and refer in the context of the present invention as any underwater ground including lakes, ponds, seas, and oceans.

In existing versions of seafloor drills, the operator is controlling the operations from a control cabin on the deck. Based on visual information from cameras the operator will control the machine and build up a pipe string, adding tools inside the drill-string by instructing the machine to perform actions via a joystick. In circumstances with bad visibility this can be very cumbersome, and operations cannot commence. The present invention addresses this issue by introducing the possibility of higher automation is brought to a higher level. By having a gripper, a storage area, and a plurality of tools, the ground testing apparatus is able to line up pipe sections and tools, connect or break drill string elements, and may be pre-programmed for the sequence of pipes and tools to be used. As a result, the operator is much less dependent on visual input from the ground testing apparatus. In a preferred embodiment, the whole sequence of drilling and testing can be pre-programmed, and the ground testing apparatus will perform the operation automatically upon receipt of a start-signal.

In the context of the present invention, the ground testing apparatus is remotely operable such as from a distance from meters up to 20000 kilometres or further away from the control location. The ground testing apparatus can be operated underwater from any depth ranging from 20 m to several kilometres such as 1 km, 2 km, 3 km, 4 km, 5 km, 6 km, 7 km, 8 km, 9 km, 10 km and/or 11 km underwater. Advantageously, the ground testing apparatus is an underwater ground testing apparatus for working at depth below the ground surface of e.g., the seabed, of more than 100 m, more advantageously of more than 150 m, still more advantageously of more than 500 m. In a preferred embodiment, the ground penetrating apparatus is an underwater ground penetrating apparatus for working at depth above 5 km, more advantageously above 4 km. Alternatively, the ground penetrating apparatus can also be used on land such as at sea level, up to 1000 m altitude, advantageously also up to 4000 m altitude.

The geometry and size of the frame of the ground testing apparatus has the advantageous ratio width/length/height of 1:1:2 which has the advantage of an increased stability in the environment in which the ground testing apparatus is operated. In a preferred embodiment, the height of the frame is less than 4 times the width and/or the length, more advantageously less than 3 times the width and/or the length, still more advantageously less than 2.5 times the width and/or the length.

In an embodiment, the frame advantageously consists of or comprises metal, advantageously steel, stainless steel, aluminium, titanium, and/or a polymer, advantageously ultra-high molecular weight polymers (such as ultra-high molecular weight polyethylene), ultra-high density polymers (such as ultra-high density polyethylene), polyoxymethylene, resins, polyamides, polyether ether ketones and polycarbonates. In the context of the present invention, the term ‘polymer’ is to be understood as a homopolymer or a copolymer. The frame may further be coated and/or heat-treated. A coating may be e.g., an abrasive-resistant coating, chemically resistant coating, oxidation-resistant coating, or a thermally-resistant coating.

The frame of the ground testing apparatus according to the present invention supports the apparatus and allows fitting into a container for transport. Advantageously, the frame has a light weight and has a stiff and strong structure to cope with the operational loads during testing and drilling. In a preferred embodiment, the ground testing apparatus has a weight of less than 15000 kg, advantageously of less than 12000 kg, more advantageously of less than 10000 kg, still more advantageously of less than 8000 kg. In a preferred embodiment, the frame has a weight of less than 6000 kg, advantageously of less than 4000 kg, more advantageously of less than 3000 kg, still more advantageously of about 2200 kg. The weights are measured above water. In the event of the apparatus deployed from a vessel in a marine environment, the frame structure also supports the loads and movements while being deployed back and forth from the vessel deck.

In the context of the present invention, the ground penetrating testing tool or tools can be any testing tools to be used on land or underwater to sample the seabed which is a pipe, a rod or a cylinder which allows penetrating and testing the ground.

Advantageously, the ground testing apparatus according to the present invention allows testing with ground penetrating testing tools of various lengths. Advantageously the length of the at least one testing tool is in the range from 1 m to 5 m, advantageously from 1.5 m to 3 m. The ground testing apparatus advantageously allows multiple testing tools to be stored, used, and moved within the ground testing apparatus according to the present invention. In a preferred embodiment, the ground testing apparatus is arranged to retrieve and store at least 20 samples, advantageously at least 45 samples, still more advantageously 60 samples. In an embodiment, the ground testing apparatus is arranged to retrieve and store at least 72 samples, advantageously at least 85 samples, more advantageously at least 90 samples, most advantageously 96 samples. The samples may be stored in the storage unit of the ground testing apparatus. The samples may be retrieved using the at least one testing tool and may be moved from the at least one testing tool to the storage unit by the gripper. The gripper is arranged to grip a sample to remove it from the testing tool, and to move it towards the storage unit.

Once the ground testing apparatus is retrieved, and is provided e.g., on the vessel from which it was deployed, according to an embodiment of the invention, the samples may be removed from the storage area and moved to a sample retrieval location, outside of the ground testing apparatus. In an embodiment of the samples retrieved by the at least one testing tool is moved from the storage unit to the sample retrieval location by a robot arm, which is advantageously provided outside the ground testing apparatus, e.g., provided on the vessel. In an alternative or additional embodiment of the invention, the samples are moved from the storage unit to the sample retrieval location by the gripper on the ground testing apparatus or by the gripper in addition to the robot arm outside of the ground testing apparatus.

In one example embodiment of the present invention, the ground penetrating tool is a device for performing a test to determine soil properties from a seabed. It may be a ground penetrating testing tool, or a tool provided in-situ sensor to determine soil parameters, such as but not limited to seismic geophones, or thermal conductivity tool or temperature. In an embodiment, the at least one testing tool comprises a cone rod unit comprising a cone rod interface unit, the cone rod interface unit comprising at least one first light source configured to generate a first light signal containing first data. The ground testing apparatus advantageously further comprises a control unit, comprising a first sensor configured to detect the first light signal containing said first data, and a communication unit configured to transmit and receive data to and from a data acquisition unit.

The at least one testing tool may further comprise one or more intermediate rod units arranged between the cone rod unit and the control unit, configured to guide said first light signal from the cone rod interface unit of the cone rod unit to the first sensor of the control unit. The cone rod unit, optional intermediate rod units, and the control unit are thus communicatively connected in an embodiment of the invention. The device is thus configured to guide a light signal from a cone rod unit to a control unit, via one or more intermediate rod units. The use of a light signal removes the need for wires, which may introduce problems with reliability.

Advantageously, the first light signal may comprise a modulated signal where the modulation is at least one of time modulation, frequency modulation, phase modulation and amplitude modulation. These are only examples of a modulation that can be used. Digital modulation is preferred as this does not suffer from degradation of the information when the signal-to-noise ratio of the channel decreases. The choice of modulation is based on efficiency (power needed for a minimum acceptable bit error rate), ease of implementation, and/or the communication channel characteristics. Advantageously, the device can be further configured such that the cone rod unit further comprises a first light guiding element, and/or each intermediate rod unit comprises a respective intermediate light guiding element. The presence of at least one such light guiding element improves the transmission of the light signal. For example, if the test string deviates from a strictly vertical alignment, the light guiding element(s) may enable the light signal to be curved, bent, or otherwise adjusted accordingly. Advantageously, at least one of the first light guiding elements and each intermediate light guiding element may be made of at least one of a glass and a polymer, such as poly(methyl methacrylate) (PMMA), glass and/or plastic. Light guiding elements made of any of these materials (or combinations thereof) are particularly suited to transmitting a light signal.

Another advantage is when at least one of the first light guiding element and each intermediate light guiding element may be coated with a light reflecting layer. The use of a light reflecting layer may further improve the transmission of a light signal, by reducing or preventing light from leaking out of the light guiding element(s). Further, the at least one first light source may comprise at least one of a light emitting diode (LED) and a laser. The use of LEDs may consume a low amount of energy and simplify maintenance and construction as LEDs are physically robust and operate predictably at a range of environmental conditions. LEDs are also small, enabling them to be fitted into a small space, and have high switching rates, which facilitates reliable signal modulation. Advantageously, the at least one LED may emit at least one of blue, violet, and green light. These colours, which are associated with wavelengths between 400-600 nm, are particularly suited to propagate through water and through any light guiding elements present. Advantageously, the cone rod unit may further comprise a lens arranged in a light path of the at least one first light source. The use of a lens may improve the propagation of the first light signal through the device by producing parallel rays, by aligning the rays with the longitudinal axis of the device.

In an embodiment, the control unit may comprise a second light source configured to generate a second light signal containing second data, and the cone rod interface unit of the cone rod unit further comprises a second sensor configured to detect said second light signal emitted by the second light source. The use of a light source at either end of the device enables bidirectional communication between the cone rod unit and the control unit, which may enable the test to be adjusted based on received signals and therefore be reactive. Advantageously, the first light guiding element and a first intermediate light guiding element of a first intermediate rod unit of the at least one rod unit may be arranged to be separated by a light coupling gap. The presence of a light coupling gap may reduce or prevent damage to the ends of the light guiding elements.

In an embodiment, the intermediate light guiding elements of adjacent intermediate rod units of the at least one intermediate rod unit may be arranged to be separated by a light coupling gap. The presence of a light coupling gap may reduce or prevent damage to the ends of the light guiding elements. Advantageously, the device may further comprise at least one repeater intermediate rod unit, which comprises a first intermediate light source disposed in a first end of the at least one repeater intermediate rod unit, an intermediate power source disposed in the first end of the at least one repeater intermediate rod unit, and a first intermediate light sensor disposed in a second end of the at least one repeater intermediate rod unit, opposite to the first end of the at least one repeater intermediate unit. The use of at least one repeater intermediate rod unit in the test string may boost or improve the transmission of the signal, especially if the signal has attenuated too much.

To automatically perform a test to determine soil properties, the ground testing apparatus may comprise a cone rod unit comprising at least one sensor and at least one light source, the at least one light source configured to generate a first light signal containing first data. The ground testing apparatus may comprise a control unit configured to be arranged at a position above a test position on a seabed at which the test is to be performed, the position and the test position defining a firing line therebetween.

The ground testing apparatus further comprises a gripper, connected to a frame, which may be configured to collect at least a first intermediate rod unit from one or more intermediate rod units from the storage unit, position the first intermediate rod unit in the firing line, directly above the cone rod unit, such that the first intermediate rod unit is axially aligned with the cone rod unit, attach the first intermediate rod unit to the cone rod unit to assemble a test string, push the assembled test string into soil of the seabed, and hold the test string during assembly. The cone rod unit is configured to transmit said first light signal to the control unit when the frame pushes the assembled test string into said soil, and the control unit is configured to sense the transmitted first light signal and communication with a remote data acquisition unit. The above-described construction is advantageous because it enables the test to be performed whilst the test string is being assembled, in an automatic fashion. In this way, the test string may be assembled at the seabed in a simple and reliable manner.

Advantageously the at least one gripper handles tools from a storage unit to a location in the apparatus where the tool will be used to perform ground penetration and testing of the ground. Consecutively, the at least one gripper handles the tools from the location in the apparatus where the tool will be used to perform ground penetration and testing of the ground back to the storage space when the testing has been performed. The at least one gripper can be one gripper or more, two grippers or more, three grippers or more.

In the context of the present invention, the least one gripper (or clamp) is arranged to handle the ground penetrating testing tools. The gripper may comprise two extending rods, arranged to grip, in a finger-like fashion, the tools/samples. The gripper is movable in three directions (X-Y-Z). It is mounted on a trolley that is supported by two rails. The trolley moves over rails that are mounted above the pipe storage units. The gripper unit is fully rotatable, allowing it to engage with a ground penetrating testing tool from different storage units and take it to the drilling firing line (or vice versa), such as a drill pipe or a CPT rod depending on the test to be performed. Tools can be picked from a carrousel type pipe storage or a more traditional fingerboard storage. The gripper is placed offset from the trolley beam and can rotate 360 degrees. This provides flexibility to pick or place items in any desired direction.

Advantageously, the gripper, connected to the frame, may be further configured to collect a second intermediate rod unit from the plurality of intermediate rod units from the storage unit, position the second intermediate rod unit in the firing line, directly above the first intermediate rod unit, such that the second intermediate rod unit is axially aligned with the first intermediate rod unit, and attach the second intermediate rod unit to the first intermediate rod unit to extend the test string. The addition of the second intermediate rod unit (and subsequent intermediate rod units) may enable the assembly and ongoing testing of soil until a desired depth is reached, and advantageously, the desired depth may be determined during the testing procedure in real time or near-real time. Advantageously, the frame may be configured to push the assembled test string into the soil at a constant speed of between 1-5 cm/s, for instance, approximately 2 cm/s.

When the ground testing apparatus of the present invention is used subsea, a practical solution to keep the rods in the storage unit during lowering through the splash zone, the water column, and landing on the seabed, is to use a carrousel plate comprising at least one lateral slot being provided above the storage unit. The provision of such a carrousel plate, prevents the pipes to move upwards. To take out pipes the slot is rotated into a position that has no cover. Alternatively, or additionally, another blocking system may be utilised such as a mechanical fixing unit such as a threaded or bayonet fixing unit. Alternatively, or additionally, a magnetic fixing unit may be utilised to stabilise and fixate the rods. The same applies to sample storage, which may also be stored and fixated by means of any of the above embodiments.

In the context of the present invention, the environment detection sensors can be any of position and/or motion sensors, pressure sensors, video cameras, magnetic, acoustic sensors used in fog, in the dark (in the absence of light), in water, in air or in absence of atmosphere.

In the context of the present invention, the wireless communication system comprises a connection tool with spearhead and blue-LED conductor.

In the context of the present invention, at least one, advantageously each, of the at least three height-adaptable legs incline to an angle up to 5 degrees, advantageously up to 10 degrees, more advantageously up to 15 degrees, still more advantageously up to 20 degrees. In an embodiment, at least one, advantageously each, of the at least three height-adaptable legs incline to an angle up to 25 degrees, advantageously up to 30 degrees, more advantageously up to 45 degrees. The advantage of the legs is that the apparatus can adapt to all sorts of ground variations such as height variations, composition variations (e.g., mix of hard and softer ground compositions), or location in a ground cavity for example.

The adaptable legs may comprise stabilizing plates or feet, but do not need such stabilizing plates or feet to suitably immobilize the apparatus because the height-adaptable legs provide suitable support to the apparatus according to the present invention. In an embodiment of the present invention, the at least three height-adaptable legs can be automated and can proceed forward on onshore or offshore ground. In other words, the apparatus according to the present invention is remotely operated to move along the ground in a three-dimensional pattern according to the ground profile.

The apparatus according to the present invention has a weight is in the range from 10000 to 17000 kg out of the water, advantageously 12000 to 16000 kg measured by conventional weight measuring techniques out of the water. The advantage of the weight of the apparatus according to the present invention is that it can be transportable on a vehicle or vessel, and it provides enough stability to the environment when in operation. A vehicle is understood to mean any potential deployment platform that moves. A vehicle may be a helicopter, a drone, a car, a truck, a ship, or the like.

In an example embodiment, the remotely operated ground testing apparatus may comprise at least two ground penetrating testing tools, advantageously at least 10 ground penetrating testing tools, more advantageously at least 30 ground penetrating testing tools, still more advantageously at least 40 ground penetrating testing tools.

In an example embodiment, at least one of the height-adaptable legs comprises a planar foot, arranged to engage with the ground, the planar foot being pivotably connected with a distal end of the at least one of the height-adaptable legs, advantageously wherein all height-adaptable legs comprise planar feet, arranged to engage with the ground, the planar feet being pivotably connected with distal ends of all height-adaptable legs.

As a result of the planar foot being pivotably connected to a distal end of at least one of the height-adaptable legs, the ground testing apparatus can be positioned on an angled surface, reducing the chance of slippage. Since the foot is pivotably connected, it can adapt to the angle of the ground when the legs are engaging therewith. As a result, the foot may be angled such that a majority of its surface engages with the ground, even if the ground is angled.

In parallel, the height-adjustable legs can extend or retract to allow the ground testing apparatus to remain level, even if the ground is not. The combination of height-extendable legs and one or more pivotably connected planar feet allows the ground testing apparatus to remain level on sloped surfaces, or stepped surfaces, and thus allows for improved positioning on various terrains.

In the context of the present invention, the ground penetrating testing tools are selected from the group consisting of drilling tools, coring tools and cone penetration testing tools. Other land and underwater testing tools may also be used in the ground testing apparatus.

In the context of the present invention, the drilling tools and the coring tools are similar tools in essence, with the different in the size of the rod used. Typically, samples can be obtained using a drill located at the lower end of a drill string. The drill string is commonly assembled from a plurality of drill rods which are connected together with threaded connections. The lowermost drill rod is known as a barrel and is comprised of an outer barrel and an inner barrel which is secured within the outer barrel at a drilling position. The drill is connected with the barrel and includes an annular cutting surface. The inner barrel collects a cylindrical sample from within the annular cut which is made by the annular cutting surface of the drill. The inner barrel contains and protects the sample.

Following the collection of a sample during drilling, the inner barrel must be removed from the interior of the drill string in order to extract the sample from the inner barrel, and a replacement inner barrel must be inserted into the interior of the drill string and secured at the drilling position in order to enable a further sample to be collected as drilling continues. In drilling, the inner barrel is removed from the interior of the drill string and the replacement inner barrel is inserted into the interior of the drill string by first removing the entire drill string from the borehole. In wireline drilling, the inner barrel is removed from the interior of the drill string without removing the entire drill string from the borehole, by using an inner barrel retrieval device such as an overshot which is attached to the end of a wireline.

The inner barrel retrieval device is inserted into the interior of the drill string and passed through the interior of the drill string on the end of the wireline until it attaches with the inner barrel. The inner barrel retrieval device and the inner barrel are then removed from the interior of the drill string by retracting the wireline. The replacement inner barrel is then inserted into the interior of the drill string and passed through the interior of the drill string until it is secured at the drilling position, either with the wireline or by pumping the replacement inner barrel through the interior of the drill string with a chaser fluid.

This process of removing an inner barrel from the interior of the drill string and inserting a replacement inner barrel into the interior of the drill string may be repeated several times or many times during the drilling of the borehole. As a result, it is apparent that an advantage of wireline drilling over conventional drilling is that wireline drilling does not require the removal of the entire drill string from the borehole each time that the inner barrel must be removed and replaced.

In the performance of land based conventional or wireline drilling, it is feasible to carry out drilling with as few as one or two inner barrels. If a single inner barrel is used, drilling must be interrupted while the inner barrel is removed from the interior of the drill string, while the sample is extracted from the inner barrel, and while the inner barrel is reinserted into the interior of the drill string. If two inner barrels are used, drilling must be interrupted while the first inner barrel is removed from the interior of the drill string and while the second barrel is inserted into the interior of the drill string, but the sample may be extracted from the first inner barrel while the second barrel is being inserted into the interior of the drill string.

The performance of underwater conventional or wireline drilling involves challenges which are not encountered in the performance of land-based drilling. For example, underwater drilling may be performed using drilling equipment which is deployed and controlled from a barge, ship or platform which is located on the surface of a body of water or may be performed using remotely operable underwater drilling equipment which is operatively connected to a barge, ship, or platform with only a deployment cable and/or a control cable.

An advantage of using remotely operable underwater drilling equipment for underwater drilling is that the underwater equipment is not generally affected by movement of the barge, ship or platform which is located on the surface so that the stability of the underwater equipment is not dependent upon the stability of the surface equipment. As a result, the underwater equipment may typically be constructed to be relatively small and light.

In the context of the present invention, using remotely operable underwater drilling equipment for underwater drilling is that although the operation of the underwater equipment may be controlled from a control location on the surface of the body of water, the entire drilling operation must be essentially self-contained and performed without physical interaction with the surface.

As one example, underwater drilling equipment carry a supply of drill rods and inner barrels which is sufficient to enable drilling to a desired depth and the collection of a desired number of samples. Consequently, a storage unit is provided on the underwater drilling equipment for e.g., a number of drill rods and inner barrels. As a second example, the underwater drilling equipment must be capable of operating remotely without manual adjustment or repair since direct human intervention with the underwater drilling equipment is not typically possible when the equipment is deployed underwater. As a result, the underwater drilling equipment and its operation are advantageously made simple and robust so that an amount of reliability in the underwater environment can be achieved.

A substantial advantage of the apparatus of the present invention is to provide an apparatus and method for penetrating and testing the ground which is sustainable because it needs fewer operators to handle the apparatus, also smaller vehicles or vessels to transport the apparatus, therefore creating a lower carbon footprint, improving safety by automation, capable of working in zero visibility, and by providing flexibility in testing tools which can be used.

In the context of the present invention, testing tools can advantageously be selected from the group consisting of a cone penetration testing (CPT) tool such as a piezocone, a seismic piezocone, a vane, a surface T-bar, a thermal conductivity cone, a temperature cone, and an electrical resistivity cone. A cone penetrometer is pushed into the ground at a defined rate. Data are recorded at regular intervals during penetration and a cone penetration test rig pushes the steel cone vertically into the ground. The cone penetrometer is instrumented in order to measure penetration resistance at the tip and friction in the shaft (friction sleeve) during penetration. The data collected depends on the ground density and chemical composition. A CPT probe equipped with a pore-water pressure sensor is called a CPTU. CPT probes with other sensors are also used.

Another aspect of the present invention relates to an automated launch and recovery system for use with, and advantageously comprising, the remotely operated ground testing apparatus as defined herein. The launch and recovery system comprises an umbilical winch with axial fleeting spooler and dedicated line tensioner, a lift winch with axial fleeting spooler and dedicated line tensioner, a telescopic A-frame and a twin line deployment system consisting of at least one lifting cable, at least one data cable and/or at least one power cable.

The telescopic A-frame has a size and mechanism suitable to lift and handle the ground testing apparatus according to the present invention. The telescopic A-frame comprises advantageously a docking head. The telescopic A-frame comprises advantageously at least one winch, at least one light and at least two telescopic arms. The telescopic A-frame can be deck-mounted, bulkhead-mounted, and roof-mounted onto a vehicle or vessel.

Yet another aspect of the present invention relates to a container for containing and immobilizing the remotely operated ground testing apparatus as defined herein, advantageously wherein the container comprises the remotely operated ground testing apparatus as defined herein.

In another aspect of the present invention, a vehicle, or a vessel advantageously comprises an automated launch and recovery system as defined in the claims and/or a lifting arrangement for the container as defined in the claims. The vehicle can be any means of transport or machine which can be used for transportation. The vessel can be a sea vessel, such as a boat or any other marine transportation mean. The vessel can also be remotely operated such as an unmanned vessel. The unmanned vessel in this context has a length between 20 m and 100 meters, similar to a manned surveying vessel. Advantageously the vessel comprises a deck robotic arm for loading and/or unloading sample tubes from the ground testing apparatus. The robotic arm is positioned on the deck of the vessel and is arranged to load and unload samples and testing equipment from the remotely operated ground testing apparatus onto the vessel. This helps reduce human interaction with the ground testing apparatus once it is hoisted on the deck of a vessel. On the deck, a barrier with a slot may be provided such that the robotic arm can provide samples through the slot of the barrier. This is to prevent that people can access the offloading station. As a result, the robotic arm clamps a sample and/or a testing tool and moves it through the slot to a safe-zone on the opposite side of the barrier with the slot.

The present invention also relates to a method for testing the ground comprising: i. providing a remotely operated ground testing apparatus according to any of the embodiments disclosed herein; ii. deploying the remotely operated ground testing apparatus so that it is positioned above ground to be tested; and iii. penetrating the ground with a ground penetrating testing tool to test characteristics of the ground and to take a ground sample of the ground. Advantageously, the sample is collected and analysed. In an embodiment, the method further comprises the step of testing one or more characteristics of the ground, and advantageously further comprises taking a ground sample of the ground to be tested. In an embodiment, the method further comprises retracting the ground penetrating testing tool from the ground.

In the context of the present invention, the method for testing the ground is a method of performing a test to determine soil properties using the apparatus described above. The method comprises holding the at least one ground penetrating testing tool (such as a cone rod unit) in the firing line, attaching a first intermediate rod unit of one or more intermediate rod units to a cone rod unit to assemble a test string, the first intermediate rod unit and the cone rod unit being axially aligned, pushing the assembled test string into the soil of the seabed, transmitting a light signal from an at least one light source of the cone rod unit to the light sensor of a control unit, and transmitting a signal from the control unit to a data acquisition unit, holding the assembled test string in the firing line, and extending the test string by attaching at least a second intermediate rod unit of the plurality of intermediate rod units to the assembled test string. This method has the advantage that it may be employed automatically and remotely, enabling the test to be performed at the seabed whilst being monitored and/or controlled remotely.

Advantageously, the operation of extending the test string may be repeated until the test string is between 20 m and 100 m in length, and advantageously between 50 m and 80 m in length. These lengths provide a suitable depth for many soil-investigation tests. Advantageously, the operation of pushing the assembled test string into the soil of the seabed may comprise pushing the assembled test string into the soil of the seabed at a constant speed of between 1-5 cm/s, for instance, approximately 2 cm/s. A constant speed in this range may provide reliable testing and facilitate the collection of reliable data

The present invention is illustrated by the following Examples and figures.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are therefore not to be considered to be limiting of its scope, the principles herein are described and explained with additional specificity and detail through the use of the accompanying drawings in which:

FIG. 1 shows a three-dimensional view of the remotely operated ground testing apparatus according to an embodiment of the disclosure;

FIG. 2 shows a three-dimensional view of a gripping system according to an embodiment of the disclosure;

FIG. 3 shows a top view of a deck robotic arm according to an embodiment of the disclosure;

FIG. 4 shows a three-dimensional view of a launch and recovery system in accordance with an embodiment of the disclosure;

FIG. 5 shows a test string during assembly according to an embodiment of the disclosure;

FIG. 6 shows a device for performing a test on a seabed according to an embodiment of the present disclosure;

FIG. 7 shows a system for performing a test on a seabed according to an embodiment of the present disclosure; and

FIG. 8 shows a flow chart illustrating a method for testing the ground according to an embodiment of the present disclosure.

### DESCRIPTION OF ILLUSTRATIVE EMBODIMENTS

The following is a description of certain embodiments of the invention, given by way of example only and with reference to the drawings.

Various embodiments of the disclosure are discussed in detail below. While specific implementations are discussed, it should be understood that this is done for illustration purposes only. Thus, the following description and drawings are illustrative and are not to be construed as limiting. Numerous specific details are described to provide a thorough understanding of the disclosure. However, in certain instances, well-known or conventional details are not described in order to avoid obscuring the description. A reference to an embodiment in the present disclosure can be a reference to the same embodiment or any other embodiment. Such references thus relate to at least one of the embodiments herein.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Without intent to limit the scope of the disclosure, examples of instruments, apparatus, methods, and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

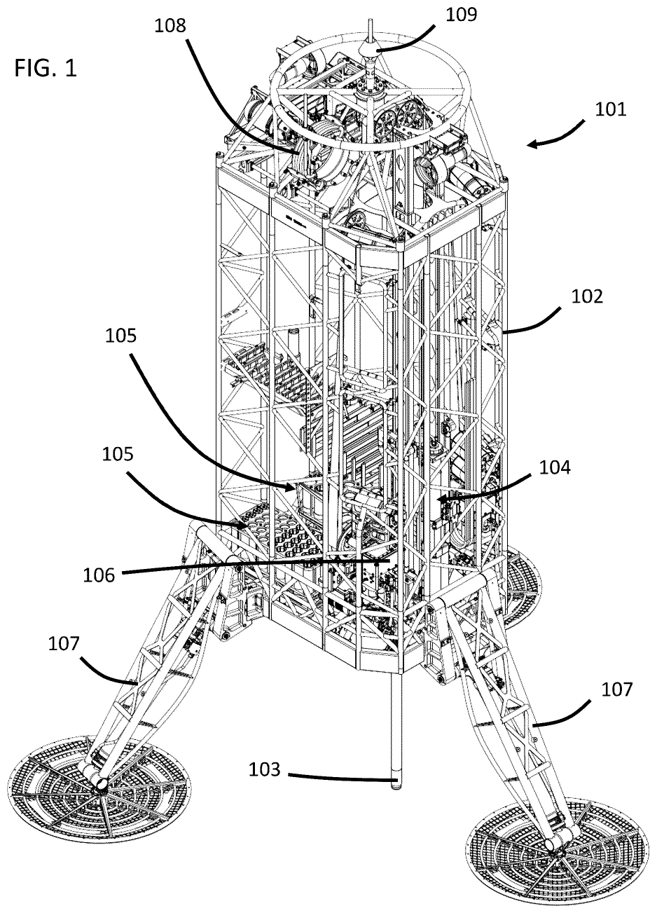

Referring to FIG. 1, a three-dimensional view of the remotely operated ground testing apparatus 101 is shown. As shown, the remotely operated ground testing apparatus 101 comprises a frame 102, at least one ground penetrating testing tool 103, at least one gripper 104, connected to the frame 102, to handle the at least one ground penetrating testing tool 103; a storage unit 105, arranged to store the at least one ground penetrating testing tool and/or testing samples, a connector 106 for connecting the at least one ground penetrating testing tool 103 to the ground testing apparatus 101, at least three height-adaptable legs 107, at least one environment detection sensor, arranged to provide environment information. The remotely operated ground testing apparatus 101 further comprises an umbilical connection 108 with axial fleeting spooler and dedicated line tensioner and a lifting connection 109 with axial fleeting spooler and dedicated line tensioner.

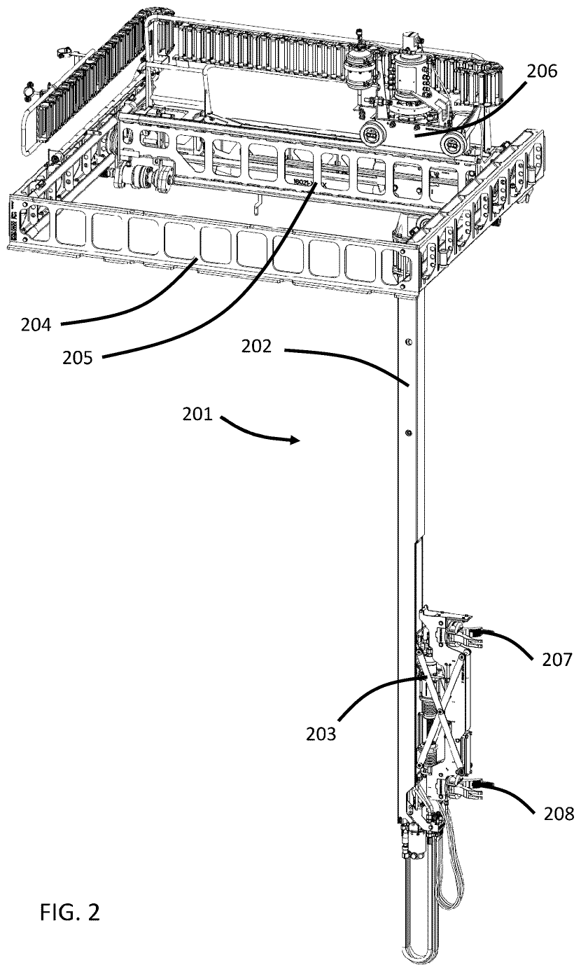

Referring now to FIG. 2, a gripping system 201 according to an embodiment of the invention is shown. The gripping system 201 comprises a vertically extending shaft 202, to which a gripper 203 is connected, such that the gripper 203 can move in a vertical direction in relation to the ground testing apparatus. The gripper 203 comprises a first 207 and a second 208 clamps, which are vertically distanced on the gripper 203. The gripper, with the first 207 and second 208 clamps is arranged to move along a vertical direction to define movement in a z-direction. The shaft 202 is also rotatable with respect to the ground testing apparatus.

The gripper 203 advantageously comprises a vertical drive system having an electronic motor, to move the gripper 203 up and down the shaft 202. The gripping system 201 further comprises a top frame 204, defining a substantially two-dimensional frame, extending in an orthogonal direction to the shaft 202 of the gripping system 201. To the top frame 204, a transverse moving beam 205 is provided, which is arranged to move along the top frame 204 in a direction orthogonal to the vertical direction of movement of the gripper 203 along the shaft 202. A trolley 206 is attached to the transverse moving beam 205, which is arranged to move along the transverse moving beam 205 in a direction orthogonal to the vertical direction of movement of the gripper 203 along the shaft 202 and the direction of movement of the moving beam 205 along the top frame 204. The shaft 202 is attached to the trolley 206. As a result, the combination of the trolley 206 and the transverse moving beam 205 provide movement of the shaft 202 in two orthogonal directions (x, y). The gripper 203 moving along the shaft in a vertical direction provides the last movement direction along the shaft 202 in the z-direction. As a result, the gripper 203 can move in three-dimensional space. The transverse moving beam 205 and the trolley 206 are driven by electronic motors, attached to the moving beam 205 and the trolley 206, respectively. The connection between the shaft 202 and the trolley 206 is a rotatable connection, wherein a rotation actuator is provided to rotate the shaft 202 with respect to the trolley. As a result, the gripper can be rotated around the shaft 202 by virtue of rotation of the shaft 202 by the rotation actuator attached to the trolley 206.

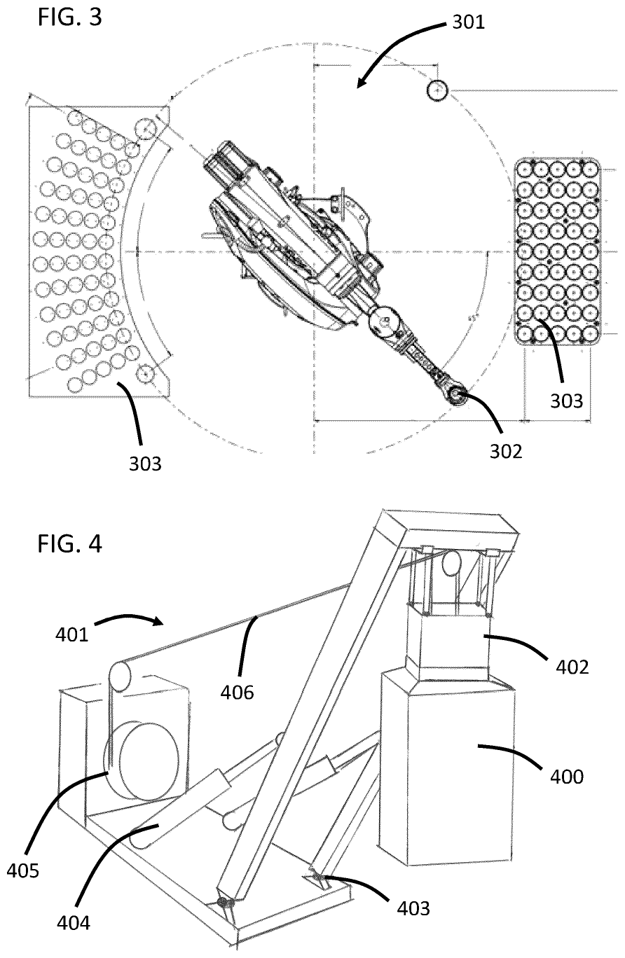

Now referring to FIG. 3, a deck robotic arm 301 is shown, comprising a clamp 302 arranged to handle samples and/or tooling. Intermediate storage areas 303 are provided on either side of the robotic arm 301, such that the tooling and samples can first be offloaded from the ground testing apparatus, prior to being handled on the vessel. The robotic arm 301 may have three degrees of freedom, advantageously six degrees of freedom. The robotic arm 301 is positioned between the ground testing apparatus and an offload area. The robotic arm 301 may retrieve soil samples and/or tooling from the ground testing apparatus which are handed off by the gripper of the ground testing apparatus.

Now referring to FIG. 4, a three-dimensional view of a launch and recovery system, preferably an automated launch and recovery system, in accordance with an embodiment of the disclosure is shown. The ground testing apparatus 400 may be lifted to or from the deck of the vessel via an A-frame 401. The A-frame 401 is pivotably mounted to the deck of the vessel and can be rotated around the pivot points 403 by extending or retracting e.g., a number of cylinders 404. Other solutions like a system with winches and wires are also possible. A lifting wire 406 is routed from a winch 405 on the vessel deck via one or more sheaves to a position below the A frame 401. The ground testing apparatus 400 is connected to the lifting wire 406. By moving the A-frame 401 inboard or outboard and spooling or unspooling the wire 406 from the winch 405, the load can be lifted from the deck, brought into an outboard position, and lowered to the seabed or vice versa.

To limit or eliminate swinging of the ground testing apparatus as an effect of vessel motions during lifting the ground testing apparatus from the position on deck to the outboard position or vice versa, a docking head 402 is connected to the A-frame 401. The top part of the ground testing apparatus 400 will be pulled into the underside of the docking head 402, and advantageously also mechanically locked onto the docking head during this operation. This can for instance be done via a spearhead on top of the ground testing apparatus, which is secured by one or more clamps in the docking head 402. Advantageously, the docking head 402 is motion compensated within the A-frame 401 via a series of hydraulic cylinders to further reduce the swinging of the ground testing apparatus 400. Once the ground testing apparatus has reached the outboard position, the mechanical lock of the docking head 402 can be opened, and the load can be lowered into the water and towards the seabed.

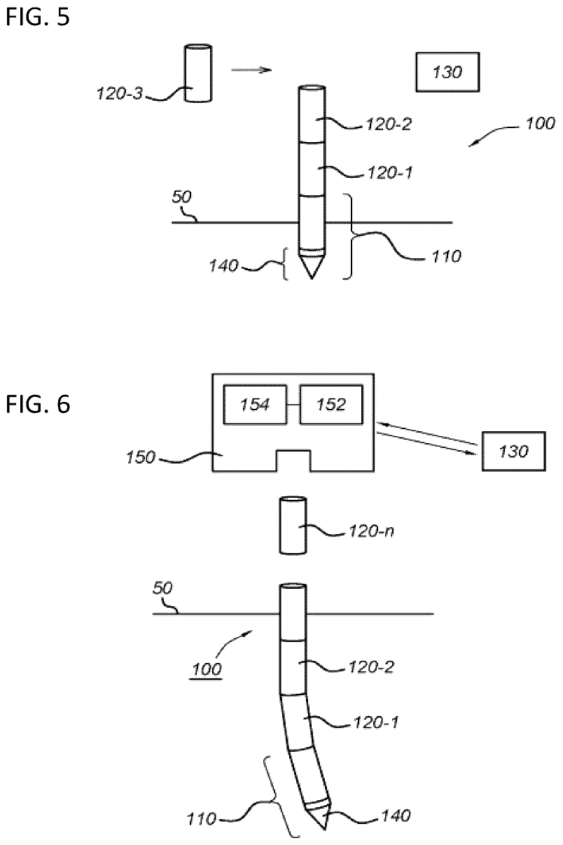

Referring to FIG. 5, a test string during assembly is shown. The test string 100 of FIG. 2 comprises a cone rod unit 110 coupled to a cone 140, and at least one intermediate rod unit 120-n (n=1, 2, . . . ) . FIG. 5 further shows a data acquisition unit (DAC) 130. The cone 140 is driven into the seabed 50 to measure soil properties. The test string 100 is lengthened by adding further intermediate rod units 120-1, 120-2 and 120-3, sequentially, until a desired depth is reached, or until further pushing is not possible, e.g., due to required force or too much deflection from a vertical plane. As the test string is lengthened, the cone 140 is driven deeper below the seabed. In an example, the test string 100 is extended to reach a length between 20 m and 100 m, for instance, between 50 and 80 m. A possible target length may be 60 m+/−10%.

The intermediate rod units 120-1, 120-2, 120-3 are added automatically to the test string. Each intermediate rod unit is added to the end of the test string opposite to the end on which the cone 140 is provided. The new intermediate rod unit 120-n is installed on top of another intermediate rod unit 120-n−1 by the gripper, which is arranged to move the intermediate rod units to the string as required. Advantageously, the remotely operated ground testing unit is provided with suitable position measurement devices to measure position and mechanical tools to perform all required mechanical actions. Such mechanical tools include the gripper, to grasp the rod units 120-n and connect adjacent rod units 120-n and 120-n−1. Then, the test string is pushed deeper into the ground.

FIG. 6 shows a device for performing a test on a seabed according to an embodiment of the present disclosure. The device comprises the assembled test string 100 as well as a control unit 150, arranged on top of the test string 100. In other words, whilst the cone 140 is arranged at a first end of the test string 100 to penetrate the seabed 50, the control unit 150 is arranged at a second end of the test string 100, opposite to the first end. The control unit 150 comprises a communication unit 152 which is configured to communicate with the data acquisition unit (DAC) 130.

In an embodiment, the communication between the DAC 130 and the communication unit 152 is wired. However, this communication may be via a wireless connection instead, such as an optical or acoustic communication system. The test string 100 is extended by adding intermediate rod units 120-n, such that the test string 100 comprises n intermediate rod units 120, n being an integer, until the desired length is achieved. As shown in an exaggerated way in FIG. 6, the cone 140 can deviate from the vertical when it is pushed into the ground 50. In practice, this deviation from the vertical position may be up to 15-20°. This can lead to a situation where a direct line of sight between the cone rod 110 and control unit 150 is no longer available. The control unit 150 is provided with a suitable processor unit schematically indicated with reference number 154, which is configured to control all actions to be performed by the control unit 150.

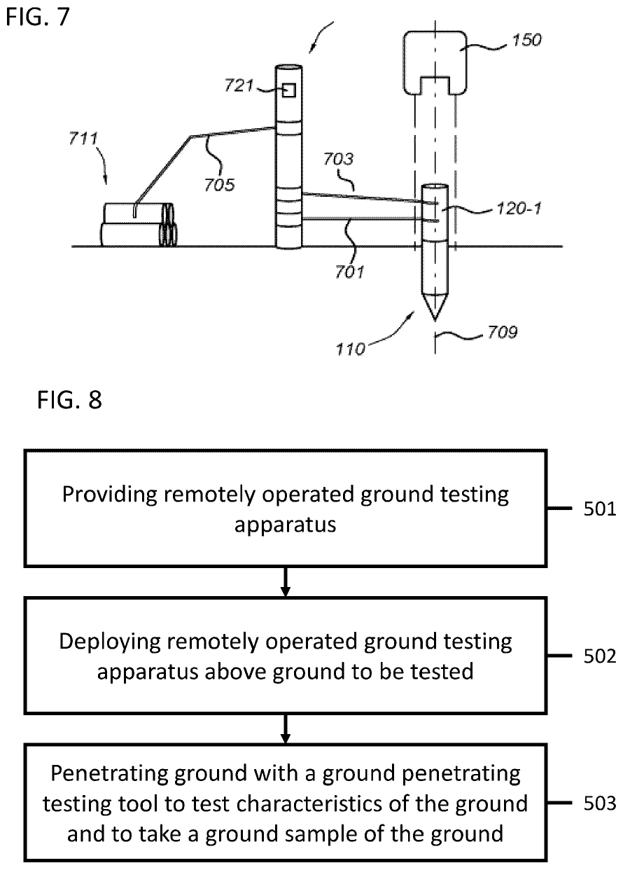

Referring to FIG. 7, a system for performing a test on a seabed is shown. The ground testing apparatus comprises a connector, such as a combination of a fixed clamp 701 and a movable clamp 703. The ground testing apparatus further comprises a gripper 705 is. The connector and the gripper are arranged as part of a frame. The frame is provided with suitable devices to control movement of the connector 703 and gripper 705. These devices may include mechanical steering mechanisms, a communication unit to communicate with a processor unit onboard of the vessel and a processor unit controlling these mechanical steering mechanisms and the communication unit. The processor unit is shown with reference number 721.

The control unit 150 is arranged above a position on the seabed at which the test will be performed. The vertical line between the test position on the seabed and the control unit 150 defines a firing line 709, which defines a line in which the string will be assembled during the test. The cone rod unit 110 is placed in the firing line 709, e.g., by means of the gripper 705 and is held in place by the connector 701. New intermediate rods 120-n (e.g., 120-1) are provided to the firing line 709 by the gripper 705, and the cone rod 110 is then pushed deeper into the ground by the control unit 150. The control unit 150 is then retracted in a vertical direction to make room for a new intermediate rod 120-n+1. After placement, the control unit 150 may again engage the rod to press the cone rod unit 110 deeper into the ground. This may be repeated as often as necessary. The intermediate rod may also be engaged differently to drive the cone rod unit 110 into the ground, for example by virtue of lateral engagement by clamping devices.

Referring to FIG. 8, a flow chart illustrating a method for testing the ground is shown. The method is shown to comprise the steps of i. providing 501 a remotely operated ground testing apparatus according to any of the embodiments disclosed herein; ii. Deploying 502 the remotely operated ground testing apparatus so that it is positioned above ground to be tested; and iii. Penetrating 503 the ground with a ground penetrating testing tool to test characteristics of the ground and to take a ground sample of the ground.

In the foregoing description of the figures, the invention has been described with reference to specific embodiments thereof. It will, however, be evident that various modifications and changes may be made thereto without departing from the scope of the invention as summarized in the attached claims.

In addition, many modifications may be made to adapt a particular situation or material to the teachings of the invention without departing from the essential scope thereof. Therefore, it is intended that the invention is not limited to the embodiments disclosed, but that the invention will include all embodiments falling within the scope of the appended claims.

Combinations of specific features of various aspects of the invention may be made. An aspect of the invention may be further advantageously enhanced by adding a feature that was described in relation to another aspect of the invention.

It is to be understood that the invention is limited by the annexed claims and its technical equivalents only. In this document and in its claims, the verb “to comprise” and its conjugations are used in their non-limiting sense to mean that items following the word are included, without excluding items not specifically mentioned. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

It will be appreciated that the present invention has been described with reference to several non-limiting exemplary embodiments and modifications can be made to the above-described embodiments without departing from the scope of the invention. Moreover, features from the above-described embodiments can be combined with other embodiments described herein.

The invention has been described by reference to certain embodiments discussed above. It will be recognized that these embodiments are susceptible to various modifications and alternative forms well known to those of skill in the art.

Although specific embodiments have been described, these are examples only and are not limiting upon the scope of the invention.

1. A remotely operated ground testing apparatus comprising:
a frame;
at least one ground penetrating testing tool;
at least one gripper, connected to the frame, to handle the at least one ground penetrating testing tool;
a storage unit, arranged to store the at least one ground penetrating testing tool and/or testing samples;
a connector for connecting the at least one ground penetrating testing tool to the remotely operated ground testing apparatus;
at least three height-adaptable legs;
at least one environment detection sensor, arranged to provide environment information.

2. The remotely operated ground testing apparatus according to claim 1, wherein the at least one ground penetrating testing tools comprises a cone rod unit, arranged to penetrate a ground, and a control unit, arranged in the frame, wherein the control unit is arranged to receive data from the cone rod unit, and wherein the data is transmitted through an optical connection.

3. The remotely operated ground testing apparatus according to claim 1, wherein at least one of the at least three height-adaptable legs incline to an angle up to 25 degrees.

4. The remotely operated ground testing apparatus according to claim 1, wherein the frame has width/length/height dimensions in a range 2 to 5 meters in width, 2 to 5 meters in length and 2 to 10 meters in height.

5. The remotely operated ground testing apparatus according to claim 1, wherein a weight of the apparatus is in the a range from 10000 to 17000 kg.

6. The remotely operated ground testing apparatus according to claim 1, comprising at least two ground penetrating testing tools.

7. The remotely operated ground testing apparatus according to claim 1, wherein at least one of the at least three height-adaptable legs comprises a planar foot, arranged to engage with the ground, the planar foot being pivotably connected with a distal end of the at least one of the at least three height-adaptable legs.

8. The remotely operated ground testing apparatus according to claim 1, wherein the at least one ground penetrating testing tool is selected from a group consisting of drilling tools, coring tools, cone penetration testing tools and underwater testing tools.

9. The remotely operated ground testing apparatus according to claim 1, wherein the remotely operated ground penetrating apparatus is an underwater ground penetrating apparatus for working at depth below 20 m.

10. An Automated launch and recovery system for use with a remotely operated ground testing apparatus, the system comprising:
a telescopic A-frame; and
a twin line deployment consisting of at least one lifting cable, at least one data cable and/or power cable,
wherein the apparatus including
a frame;
at least one ground penetrating testing tool;
at least one gripper, connected to the frame, to handle the at least one ground penetrating testing tool;
a storage unit, arranged to store the at least one ground penetrating testing tool and/or testing samples;
a connector for connecting the at least one ground penetrating testing tool to the remotely operated ground testing apparatus;
at least three height-adaptable legs;
at least one environment detection sensor, arranged to provide environment information.

11. (canceled)

12. The automated launch and recovery system according to claim 10, further comprising an umbilical winch with axial fleeting spooler and dedicated line tensioner and a lift winch with axial fleeting spooler and dedicated line tensioner.

13. (canceled)

14. The automated launch and recovery system according to claim 12, wherein the system is coupled to a vessel, the vessel including a lifting arrangement, arranged to lift a container for containing and immobilizing the remotely operated ground testing apparatus.

15. A Method for testing a ground comprising:
providing a remotely operated ground testing apparatus, the apparatus including:
a frame;
at least one ground penetrating testing tool;
at least one gripper, connected to the frame, to handle the at least one ground penetrating testing tool;
a storage unit, arranged to store the at least one ground penetrating testing tool and/or testing samples;
a connector for connecting the at least one ground penetrating testing tool to the apparatus;
at least three height-adaptable legs;
at least one environment detection sensor, arranged to provide environment information;
deploying the remotely operated ground testing apparatus so that it is positioned above ground to be tested; and
penetrating the ground with a ground penetrating testing tool to test characteristics of the ground and/or to take a ground sample of the ground.

16. The automated launch and recovery system according to claim 10, wherein the at least one ground penetrating testing tool comprises a cone rod unit, arranged to penetrate a ground, and a control unit, arranged in the frame, wherein the control unit is arranged to receive data from the cone rod unit, and wherein the data is transmitted through an optical connection.

17. The automated launch and recovery system according to claim 10, wherein at least one of the at least three height-adaptable legs incline to an angle up to 25 degrees.

18. The automated launch and recovery system according to claim 10, wherein the frame has width/length/height dimensions in a range 2 to 5 meters in width, 2 to 5 meters in length and 2 to 10 meters in height.

19. The automated launch and recovery system according to claim 10, wherein a weight of the apparatus is in a range from 10000 to 17000 kg.

20. The automated launch and recovery system according to claim 10, comprising at least two ground penetrating testing tools.
